(19) United States
(12) Patent Application Publication
(10) Pub. No.: US 2011/0136262 A1
(43) Pub. Date: Jun. 9, 2011

(54) INTEGRATED MICROCHIP SENSOR SYSTEM FOR DETECTION OF INFECTIOUS AGENTS

(75) Inventors: Vanaja Vijaya Ragavan, Brynn Mawr, PA (US); Avijit Roy, Springfield, PA (US); Herman Rutner, Hatboro, PA (US)

(73) Assignee: Aviana Molecular Technologies, LLC

(21) Appl. No.: 12/790,475

(22) Filed: May 28, 2010

Related U.S. Application Data
(60) Provisional application No. 61/182,646, filed on May 29, 2009.

(51) Int. Cl. G01N 33/543; G01N 27/00

(52) U.S. Cl. 436/518; 422/69

(57) ABSTRACT

An integrated multiplexed acoustic wave biosensor chip system with enhanced sensitivity has been developed. The biosensor system incorporates one or more microfluidic channels, coated with target-specific binding films enabling rapid and early detection of viral, bacterial or parasitic targets such as Dengue virus and sexually transmitted diseases in specimens from potentially infected patients. The biosensors are used in portable analytical systems that are suitable for real-time point of care (POC) clinical diagnosis in cost sensitive and/or resource limited settings. The highly sensitive biosensors utilize thinned single crystal piezoelectric substrates that propagate layer guided shear horizontal acoustic plate mode (LG-SH-APM) waves in sensing regions bearing immobilized binders that provide simultaneous and direct detection of mass changes due to multiple bound target pathogens or molecules.

### CROSS REFERENCE TO RELATED APPLICATIONS

This application claims benefit of and priority to U.S. Provisional Patent Application No. 61/182,646 filed on May 29, 2009, and where permissible is incorporated by reference in its entirety.

### FIELD OF THE INVENTION

The present invention relates generally to microchannel multiplexed biosensors using piezoelectric surface acoustic wave technology and in particular to apparatus, systems, kits, collection methods, software and hardware technologies and microfluidics, devices, and methods using multiple acoustic tracks for rapid detection of infectious agents and derived toxins or proteins in biological samples of potentially infected patients or animals.

### BACKGROUND OF THE INVENTION

Biosensor technologies have tremendous potential to positively impact human health and veterinary medicine. They can be cost-effective point of care (“POC”) clinical diagnostic tools that can be deployed rapidly when needed and are useful in both the developed world and in resource limited settings. No other area could benefit from such a tool as much as the detection and treatment of acute infectious diseases affecting humans and animals. There are innumerable infections that plague humans and animals, which if diagnosed accurately at the point of contact with a health care worker, could be treated to help heal infections and decrease spread of disease. Such infections can be found uniquely or in association with many other correlated infections. Many other such acute infections may also be diagnosed alone or together in other clinical situations.

One critically important example is sexually transmitted infections (STIs), which can be caused by multiple infectious agents, such as Chlamydia, gonorrhea, and others, all in the same patient. In the example of STIs, prompt detection and treatment of curable STIs can also lead to reduced prevalence of incurable STDs such as HIV. The association between non-ulcerative and curable STIs such as gonorrhea and Chlamydia and subsequent infection with incurable STDs such as HIV has become better recognized in recent literature. This association is especially clear in the female population, and timely treatment of curable STIs can lead to reduction in HIV infection and other incurable STDs.

Clearly, there is a significant unmet need to determine the presence of STD/STIs at the time of a patient's initial visit to the physician for any reason, if there is any suspicion that the patient may be at high risk for STIs. Both screening of asymptomatic subjects and evaluation of symptomatic subjects would result in more rapid diagnosis and treatment. As part of the World Health Organization (WHO) 2001 Sexually Transmitted Diseases Diagnostic Initiative, the organization explored the need for simple, affordable, point-of-care (POC) STD testing for curable bacterial STIs, including syphilis, gonorrhea (“GC”) and Chlamydia (“CT”). The focus of this initiative was the need in the developing world for the diagnosis and treatment of these common STIs in a single health care visit through the use of rapid testing. As the authors in the cited study state: “Diagnosis and treatment in a single visit is an important step in infection control in areas where limited health care facilities and limited means of transportation can make arranging visits for tests difficult and therefore, receiving treatment improbable”. While this need is particularly dire in resource limited settings, such a proposition is also very applicable to developed nations such as the United States, where a common problem in STD clinics is patients who present for testing, but never return for follow-up.

The current marketplace does not provide adequate POC testing that is clinically useful even though there are several commercially available point of care tests for sexually transmitted diseases. Inverness Medical makes the BioStar test for CT and the Clearview test for GC. Quidel makes the Quick-Vue CT test. However, their usefulness is limited due to low sensitivity. Currently available POC tests diagnose only 2 of 3 infections, providing a false negative rate of greater than 30%. For that reason, the primary diagnostic tool is a lab based Nucleic Acid Testing (NAT), such as Aptima (Genprobe), ProbeTec (BD), and Amplicor (Roche), three widely available nucleic acid based lab tests for chlamydia and gonorrhea. Table 1, using data from Greer, et al. Inf. Dis. Clin. of N. America 22:601-617 (2008), show that nucleic acid amplification tests (NAATs) have very high sensitivities and specificities. However, NAAT testing is too costly and complex for use by minimally trained personnel. The typical four-hour time required to conduct these tests also does not include the time to transport the samples to the lab, and the real-time required for test results to be returned to remote labs, often several days. By comparison, the currently available POC immunoassay-based tests demonstrate sensitivity that is unacceptably low, hence not widely used in the marketplace.

Table 1 below shows the sensitivity and specificity of currently available tests:

Characteristics of Diagnostic Tests for Gonorrhea in women (from Greer et al. [1])**

| | | | Nucleic Acid | | | Immuno- | |
|---|---|---|---|---|---|---|---|
| Test | Bacterial | | Amplification and | | Optical | chromatographic | |
| Characteristic | Culture | Microscopy | Hybridization | | Immunoassay | Strip | |
| Specimen | Endo | Endo | Endo | Urine | Endo | Encdo | Vaginal |
| | cervical | cervical | cervical | | cervical | cervical | |
| Sensitivity | 60-95% | 50% | 92%-97% | 64%-100% | 60% | 70% | 54% |
| Specificity | 99%-100% | >95% | 98%-100% | 98$-100% | 90% | 97% | 98% |
| Ease of Use³ | +++ | ++ | +++ | +++ | ++ | ++ | + |
| Time | 48 h | 1 h | >4 h | >4 h | <30 min | <30 min | <30 min |

| | | | | | |
|---|---|---|---|---|---|
| Average Cost^{b} | $$ | $ | $$$ | $$$ | Information not available |

³Ease of use ranked (+), minimal equipment and training required (++++), highly trained personnel and sophisticated equipment required.
^{b}Average cost ranked from $ low cost to $$$$ highest cost test to perform
**Adapted from Herring A., Ballard R., Mabey D. et al. Evaluation of rapid diagnostic tests: Chlamydia and gonorrhea. Nat. Rev. Microb. 2006: Suppl. S: 42

None of the tests above meet the criteria set by the WHO, hence an major unmet clinical need remains.

Another important medical need is exemplified in rapid POC diagnosis of acute or early stage viremia of dengue fever that would limit the spread of infection and thus control epidemic outbreaks. Dengue Fever (DF) is a disease caused by a family of arboviruses (also called arthropod-borne viruses) which are transmitted by mosquitoes. A number of Aedes (Stegomyia) spp., including Aedes egyptii, Aedes, albopictus, Aedes polynesiensis and other members of the Aedes scutellaris group may act as vector, depending on the geographic area (Gubler, In T.P. Monath (ed), Epidemiology of Arthropod-borne Viral Disease. CRC Press, Inc. Boca Raton, Fla. 1988 p. 223-260). The dengue virus harbors single-stranded RNA. It has four antigenically distinct serotypes known as DEN-1, 2, 3 and 4 (Westaway, et al., Intervirology, 24:183-92 (1985); Lindenback and Rice, In Knipe, D M: Howley, P M, eds. Fields Virology. Fourth ed. Vol 1. Baltimore: Lippincott Williams and Wilkins; 2001 p. 963-1041). All serotypes can cause the full disease spectrum of dengue, which can present with undifferentiated febrile illness leading to classic dengue fever (DF), potentially fatal dengue hemorrhagic fever (DHF) or dengue shock syndrome (DSS) (Burke and Monath, In: Knipe, D M, Howely P M eds. Fields Virology. Fourth eds. Vol 1: Lippincott Williams and Wilkins; 2001. p 1043-1125). Infection with one dengue serotype provides lifelong immunity to that serotype, but there is no cross-protective immunity to the other serotypes and a second infection can cause severe disease, because the antibodies formed for one serotype do not neutralize other serotypes and can augment the infection.

According to a World Health Organization's report on Dengue fever causes tens of millions of DF cases and hundreds of thousands of DHF/DSS cases annually worldwide (WHO. Dengue haemorrhagic fever: diagnosis, treatment, prevention and control, 2^{nd} edition. Geneva: World Health Organization. 1997). A pandemic of dengue began in Southeast Asia after World War II and has spread around the globe since then, the spread being accelerated by intercontinental air travel. This pandemic also caused a rise in multiple serotypes (hyperendemicity) in a single population. Such epidemics caused by multiple serotypes are becoming more frequent, the geographic distribution of dengue virus and their mosquito vectors having expanded, with DHF emerging in the Pacific region and the Americas (Gubler, Clin. Microbial. Rev., 11(3):480-496 (1998)). By 1975 it had become a frequent cause of hospitalization and death among children in many countries (CDC Dengue Fever Fact Sheet). While a first infection by a single serotype may not cause major morbidity or mortality, reinfection by a second serotype often causes a hyper-immune reaction and can result in a significant increase in morbidity and mortality. Currently, DF and dengue infestation has become worldwide, with about 2.9 billion people at risk.

The most vulnerable time for transmission of the dengue virus is confined to a specific phase in its growth cycle. The Dengue virus has two growth cycles, one within the human and one within the mosquito Aedes aegyptii, as shown in FIG. 1A. After a first infected person or host is bitten by a mosquito during the first five days after infection, the virus incubates in the mosquito for approximately a week, when it becomes a vector for infection of a second person by mosquito bite. Following an incubation period of about a week, the second host becomes viremic over a period of about five days during which a female Aedes aegyptii mosquito biting the second person ingests blood containing the dengue virus and becomes a carrier. The newly infected mosquito spreads the disease to all bite victims during its lifetime. The most vulnerable time for spreading dengue virus is during the days of active viremia which coincides with the onset of fever, as shown in FIG. 1B (Vaughn et al., J. Infectious Diseases, 176:322-330 (1997)). Therefore, the best means to prevent the spread of infection is to quarantine the infected patient from further mosquito bites during the days of fever when viremia is at its highest. By the time the fever subsides, the viremia is also gone and the patient is no longer infectious to a biting mosquito. The major limiting factor to unequivocal diagnosis is the lack of or availability of a simple POC diagnostic test that would detect viremia during the first few days, and that is also inexpensive and affordable in a resource limited settings

Five basic antibody based serologic tests have been or are currently used for diagnosis of dengue infection: hemagglutination-inhibition (HI), complement fixation (CF), neutralization tests (NT), immunoglobulin M (IgM) capture enzyme-linked immunosorbent assay (MAC-ELISA) and indirect immunoglobulin G ELISA. HI antibody usually begins to appear at detectable levels by day 5 or 6 of illness. The major disadvantage of the HI test is its lack of specificity, which generally makes it unreliable for identifying the infecting virus serotype. The CF test is not widely used for routine dengue diagnostic serologic testing since it is difficult to perform, and requires highly trained personnel. The CF antibody generally also appears later than the HI antibody (Gubler, In T.P. Monath (ed), Epidemiology of arthropod-borne viral disease. CRC Press, Inc. Boca Raton, Fla. 1988 p. 223-260). The MAC-ELISA test has become the most widely used serologic test for dengue diagnosis. It is a relatively simple and rapid test requiring very little sophisticated equipment or user skills. However, because of the persistence of the IgM antibody for 1 to 3 months, MAC-ELISA positive results obtained with a single serum sample are only indicative of past and not necessarily recent dengue infection. Similarly, a negative result may be a false negative because the sample was taken before detectable IgM appeared (Gubler, Clin. Microbial. Rev., 11(3):480-496 (1998)). The IgM antibody only reaches levels that are considered positive 2 to 3 days after a virus induced fever falls below 38° C. (See FIG. 1B), thus providing a relatively narrow diagnostic window. The IgG-ELISA is very non-specific, exhibiting the same cross-reactivity among flaviviruses as the HI test. Other available technologies for diagnosing dengue include reverse transcriptase PCR (U.S. Pat. Nos. 7,041,255 and 6,333,150), hybridization probes (reviewed in Gubler, Clin. Microbial. Rev., 11(3):480-496 (1998)) and detection of dengue virus using magnetic separation and fluorescence (Chang, et al., Analyst, 133:233-240 (2008). However, the difficulties of working with RNA and the technical expertise required to obtain reproducible results make these methods more suitable as research tools than as routine POC diagnostic tests in a field setting.

Thus, there is clearly an unmet need for a point of care test allowing rapid and specific detection of the dengue virus in viremia, usable in tropical climates and also not requiring high technical skills and costly instruments or reagents. It is also important to know which serotype is present to assure that the patient is not at risk for a more severe course of disease. Furthermore, rapid transmission of data to a public health agency can also result in measures to reduce mosquito populations, specifically in areas of diagnosed infection.

Similar needs exist with other infectious disease agents. For instance, the influenza virus is known to mutate on a seasonal basis. The ability to quickly develop and disseminate a diagnostic tool to diagnose varying mutations is critical to reduce spread and treat influenza on a year by year basis. In another example, there continues to be an evolution of drug resistance in a number of infections. Inexpensive identification of carriers can also assist in decreasing spread of infection. Mycobacterium tuberculosis, Neisseria gonorrhea, and staphylococcus aureus are examples of organisms that have developed drug resistance. In many instances, such as drug-resistant Staphylococcus aureus or MRSA (“multi-resistant Staphylococcus aureus”), an immediate POC diagnosis at the time of a patient's emergency room visit may allow rapid start of proper treatment and prevent spread to vulnerable populations in the hospital. With regard to tuberculosis, the high prevalence in resource-limited settings demands an accurate diagnosis to identify, treat and monitor the patients with increasingly common drug resistant tuberculosis.

In summary, a highly sensitive and easy to use POC diagnostic test methods would have significant utility in both resource limited and well funded settings by not only allowing prompt and proper patient care, but also addressing public health implications for prevention of epidemics and the spread of drug-resistant diseases.

It is therefore an object of the present invention to provide efficacious and cost-effective POC diagnostic tools for detection of diverse pathogens of bacterial, viral or parasitic origins and their associated toxins.

It is an object of the present invention to provide efficacious and cost-effective POC diagnostic tools for detection of the dengue virus to enable active infections to be accurately diagnosed at the time of first visit and, a few days later, allowing monitoring of changes in infectivity.

### SUMMARY OF THE INVENTION

Multiplexed acoustic wave array biosensor systems with enhanced sensitivity incorporating multiple microfluidic channels coated with films of biologically specific binders have been developed, thereby enabling rapid direct early detection of toxins or intact organisms of bacterial, viral or parasitic infectious agents such as sexually transmitted agents, influenza or Dengue Virus in blood, serum or other body fluids of potentially infected patients. Also provided is a biosensor based method for early detection of multiple serotypes or strains of infectious agents, for example, detecting all four serotypes of the dengue virus or multiple strains/infectious agents of sexually transmitted diseases, or infectious agents which are known to cause infections by release of toxins, contain drug resistance mutations or cause cancer.

The biosensor system consists of an actively coated biosensor along with microfluidics that assist in delivering biological samples, a waste containment unit, a portable reader with the ability to transmit data wirelessly, and other reagents necessary to process biological samples. Also provided is a portable diagnostic system for real-time point of care clinical diagnosis suitable for use in applications that could be cost sensitive and/or in resource limited settings.

In one embodiment, the diagnostic systems include a reusable portable reader capable of simple push-button operation for automated analysis of samples with optionally embedded GPS systems and/or wireless systems to transmit data to public health agencies or central laboratories. In a preferred embodiment, the enhanced sensitivity sensor arrays utilize thinned single channel crystal piezoelectric substrates that propagate layer guided shear horizontal acoustic plate mode (LG-SH-APM) waves in sensing regions on multiple on-chip microfluidic channels with individual biologically specific coatings to provide simultaneous direct identification of multiple serotypes or strains. In the most preferred embodiment the piezoelectric substrate is lithium niobate processed as described in U.S. Pat. No. 7,500,379. This provides a rapid and sensitive POC multiplexed biosensor device system based on acoustic wave changes which is functionalized for binding and detection of specific markers for early detection of bacterial, viral and parasitic infections found in the biosensor component of the device. The multichannel biosensor and methods of use thereof can simultaneously detect multiple serotypes/resistant factors/pathogens during infections potentially present in a single patient, such as all four serotypes of the dengue virus or multiple STDs present in a patient together.

### BRIEF DESCRIPTION OF THE DRAWINGS

FIGS. 1A and 1B show the life cycle and routes of transmission of the Dengue virus by its insect vector Aedes aegyptii (FIG. 1A), and the relationship between viremia, fever, and DEN antibody production in infected humans (FIG. 1B).

FIGS. 2A-D are front (2A,B-electronic) and back (2B,C-biological sample) views of a two channel array chip, one reference and one active and its various components.

FIG. 3 is a cross sectional view of the two channel chip of FIGS. 2A-D.

FIGS. 4A and 4B are schematic drawings of a five channel chip for the detection of the four serotypes of Dengue Fever with a reference channel and five channels which can each be separately coated with different nanostructured bioreceptors. FIG. 4A is the back view where the biological samples are transmitted through the channels. FIG. 4B is the front view with varying arrays of transducers.

FIG. 5 is a cross-sectional view of a contained biosensor system with multiple testing channels capped for clinical use.

FIG. 6 is a prospective view of a handheld analytical device to read the biosensor results and wirelessly transmit the results.

FIG. 7 is a schematic of a point of care detection system, with the biosensor integrated into a microfluidic system, appropriate reagents, a hand held reader with wireless communication capability.

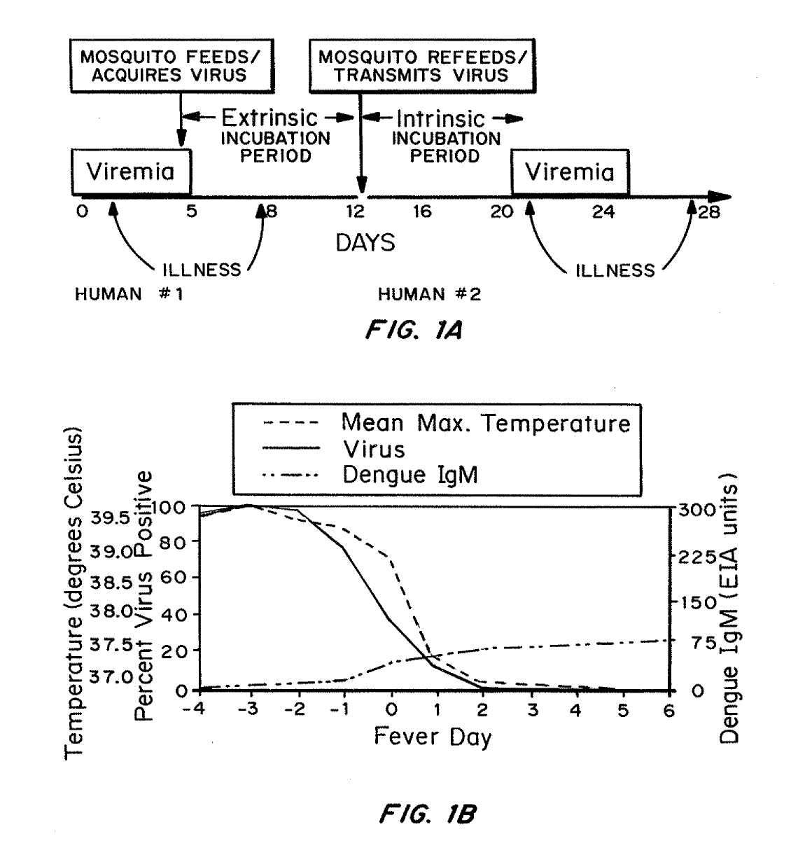

### DETAILED DESCRIPTION OF THE INVENTION

A. Biosensor Array Chip Design

A rapid, accurate and portable diagnostic system based on piezoelectric biosensors has been developed. Piezoelectric sensors have been described for laboratory based or non-commercial bioanalytical applications, including detection of infectious agents and other molecules, and for the direct real-time monitoring of affinity interactions, further including determination of the kinetic rate constants for the interactions (Skládal, J. Braz. Chem. Soc., 14: 491-502 scielo (2003)). As used herein, “infectious agents” include bacteria, viruses, toxins, parasites, virions, and infectious intermediary bodies. Most piezoelectric biosensors detect mass changes induced by formation of biocomplexes at the sensor surface, although biosensors using changes in fluid viscosity and sensors responding to changes in electrical conductivity have also been investigated. Bioreceptors have also been immobilized on surfaces of various piezoelectric devices in laboratory instruments for direct detection of analytes, including antibodies, proteins, DNA and RNA, and other large molecules.

Piezoelectric and acoustic devices such as quartz crystal microbalance (QCM) and surface acoustic wave (SAW) biosensors provide research tools for direct analyses as alternatives for more complex optical detection techniques, including surface plasmon resonance (SPR), fluorescence assays, mass spectrometry, etc. These research tools are rarely used as biosensor devices by manufacturers of clinical instruments, in part due to high costs, or scaling/manufacturing complexities, and the need for skilled operators, and are therefore not suitable for low cost POC applications in remote settings.

Research results have demonstrated feasibility but not commercial practicality of piezoelectric bioaffinity sensors as tools in the rapid detection of bacteria, viruses, proteins, nucleic acids, and other biologically relevant targets, with sensitivities that can meet or exceed alternative detection approaches. Similar results were obtained with QCM devices and other piezoelectric biosensor devices utilizing various acoustic wave propagation modes, including shear horizontal surface acoustic waves (SH-SAW), and acoustic plate modes (APMs). It is well known that the higher operating frequency of SAW based devices can provide substantial increases in sensitivity relative to bulk devices such as the QCM for which the crystal thickness sets the operating frequency, meaning that as operating frequency increases, the crystal substrates become thinner and more fragile, thus limiting QCM devices to low and sub-optimal operating frequencies of 5-10 MHz. A modified piezoelectric biosensor using surface acoustic waves based biosensor integrated into a system for a rapid diagnosis at the point of care has therefore been developed.

Acoustic wave array sensors for detection of gaseous chemicals is described in U.S. Pat. No. 6,571,638 and is proposed for use as biological sensors or biosensors using aqueous reagents for detection of bacterial, viral or parasitic infection in biological specimens in U.S. Pat. No. 7,500,379 to Hines. Hines proposes acoustic wave arrays for detection of chemicals and biologics in specialized films, wherein the arrays are capable of differentiating target molecules based on size or shape, both in gaseous and aqueous biological samples. However, there are substantial differences in film characteristics for detection of small gaseous molecules, depending largely on reversible diffusion into regenerable stable reusable films, compared to single use protein based binders or films. Selection of target specific films for biosensors is far more complex and highly critical to successful biosensor development for detection of infectious agents, particularly biosensors for large molecules or particles including, but not limited to, nanometer or micrometer sized pathogens, toxins, and drug resistance factors. Critical differences include sensor types and specificities, sensor film deposition and adhesion, strict isolation of electronic and fluidic compartments, thermal and storage stabilities of biosensors, robustness of deposited films, reproducibility of film deposition, minimization of non-specific binding (NSB) of non-target species, highly complex matrices such as blood and other relevant biologically derived fluids, and compatibility with processing steps, that have to be optimized in biosensor applications involving analysis in far more highly complex biological fluids. Conventional shear horizontal surface acoustic wave (SH-SAW) and acoustic plate modes (APMs) devices fabricated on standard wafer thicknesses, similarly, have not demonstrated theoretical attainable sensitivity for a given device operating frequency, and various means of optimally localizing the acoustic wave at the surface of the sensors have been investigated to provide enhanced device sensitivity.

A further impediment to the widespread commercial use of existing SAW-based biosensors is the requirement for a cost-effective, manufacturable detector chamber or sample compartment allowing controlled fluid flow onto the sensor surface while avoiding spillover or leakage into the electronic compartment, hence interference with the propagation of the acoustic wave. For both SAW and APM devices, relatively complex leak proof packaging is required to ensure separation of electronic from fluid handling components, using features such as spring loaded electrical contacts and rubber seals to ensure liquid tight properties for the fluid cell (Teston, et al, IEEE Trans Ultrason Ferroelectr Freq Control, 45:1266-72 (1998)).

Operating frequencies for SH-SAW and APM devices are determined not only by the crystal thickness, but by electrode periodicity, meaning that thicker, less fragile crystal substrates are usable. These devices with thick crystals can be readily fabricated to exhibit fundamental mode operation from 70 MHz to over 2 GHz. SH-SAW immunosensors operating at 345 MHz were shown to have attained the theoretical mass detection limit of approximately 33 pg and a response sensitivity of 110 kHz/(ng/mm²). However, traditional SH-SAW and APM devices on standard wafer thicknesses cannot achieve the highest possible sensitivity for a given operating frequency. Various means of localizing the acoustic wave to the surface of the device have been investigated to improve device sensitivity, such as surface transverse waves (STWs) that can be formed when wave guiding structures trap the propagating wave close to the surface. Use of a layer with lower shear acoustic speed than the substrate will result in so-called Love waves, which are also trapped at the interface between the substrate and the layer. In practical terms both of these approaches localize the acoustic wave at the sensor surface to maximize placement of the acoustic wave as close as possible to the attachment plane of the bioreceptors or binders, thereby increasing the mass sensitivity of the device. Theoretical analysis of the mass sensitivity of these devices, however, suggest that maximum mass sensitivity should occur when the biosensor device is operating at a point where the propagating wave is on the verge of transitioning from propagation inside the substrate to propagation in the overlaying layer, which would produce a larger change in phase shift of the wave with a smaller amount of added mass. LG-SH-APM devices based on this concept are described in U.S. Pat. No. 7,500,379, utilizing thinned channels on piezoelectric single crystal substrates to provide flexibility in adjusting the device substrate thickness to allow production of layer-guided SH-APM sensors with high mass sensitivity, while maintaining the structural integrity of the surrounding wafer to allow for robust device handling and packaging. These devices should exhibit enhancement of mass sensitivity by a factor of approximately 10¹-10⁴ as a possible range, compared to conventional non-layer-guided SH-APM devices, thus enabling detection limits of approximately 0.1-100 pg, or about at least 30 times lower mass than conventional SAW devices. For comparison, the device in Ben-Dov using SAW detection provides much lower sensitivity comparable to lab based ELISA testing (Ben-Dov I. et al. Anal. Chem. 1997;69(17): 3056-3512) and far lower than the biosensor chip described in U.S. Pat. No. 7,500,379 using thinned channels. Such thinned channel SAW devices suitable for high volume manufacturing at relatively low cost enable manufacture of relatively inexpensive yet extremely sensitive biosensor devices needed for detection of potential infectious agents and toxins in resource limited settings.

B. Microfluidic Channels Contained in the Biosensor Array

The biosensors or chips range in size from 100×100 mm, preferably 10×20 mm and more preferably 5×15 mm, to accommodate 2 to 6 or more separate channels per chip or two or more chips individually and combined in an integrated biosensor chip. If the chips contain channels, the channel widths can be 0.1 to 5 mm, preferably 0.5 to 2 mm and more preferably 0.2 to 1 mm. Their lengths can vary from 1 to 100 mm, preferably 5 to 20 mm, more preferably from 10 to 15 mm accommodate sample volumes of 1 to 1000 μL, preferably 5 to 100 μL and more preferably 10 to 50 μL. However, there is no particular limit to the number of channels that can be etched on an array chip.

In one embodiment, the chip utilizes a conventional piezoelectric niobate wafer that can be cut and etched or grooved to provide multiple channels per chip. Piezoelectric substrates which are useful include tantalate and silica as well as the preferred material, lithium niobate (LiNbO₃). In a preferred embodiment, a conventional 0.05-0.5 mm thick LiNbO₃ wafer, polished on both sides, is used as the piezoelectric substrate. The crystal cut is selected for both good wave propagation and etch characteristics. Such wafers meeting electronics/SAW industry standard specifications can be purchased from commercial vendors, and processed by cutting to proper size and then using thermal inversion and etch process described in U.S. Pat. No. 7,500,379 to produce biosensor chips of the selected design and dimensions.

The biosensor system includes multiple target specific channels for both capture and detection of one or more target analytes such as pathogens in a single sample potentially containing multiple strains, serotypes, drug resistant strains, various toxins as well as negative and positive controls.

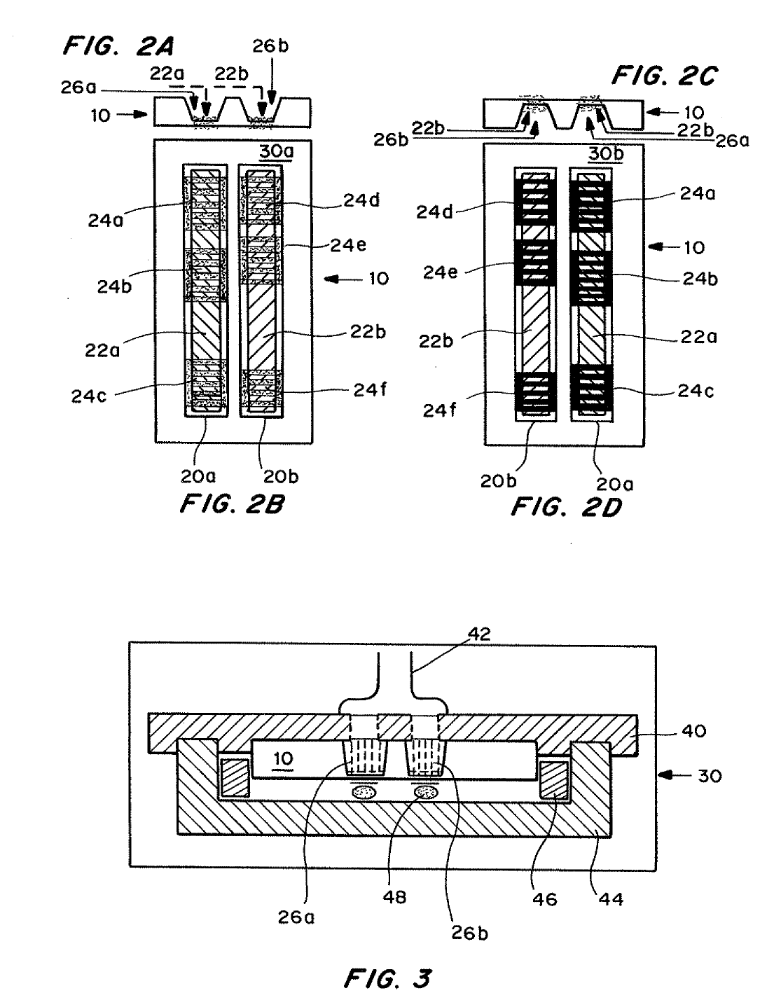

In one embodiment, in its simplest form, an array chip can be configured with two channels, a testing channel to detect an infection like Chlarnydia trachomatis and a reference channel. A schematic drawing of the sensor chip is shown in FIGS. 2A, 2B, 2B and 2C. FIGS. 2A and 2B show a cross sectional view (FIG. 2A) and top view (FIG. 2B) of the sensing side 30a of the sensor chip. The chip 10 is made of a piezoelectric substrate into which, in this example, are etched two microfluidic channels, one 26a which serves as the active channel and the other 26b which serves as the reference channel etched at the bottom of the chip. The active channel 26a is functionalized with antibody to form a bioreceptor layer 22a and the reference channel 26b is coated with a molecule which is non-reactive or measures background binding 22b. FIGS. 2C and 2D show a cross sectional view (FIG. 2C) and top view FIG. 2D) of the active electrical side 30b of the chip 10.

Referring to FIG. 2A-2D, each channel (26a and 26b) has multiple metal electrode structures (24a, 24b, 24c, 24d, 24e and 24f) on the active electrical side 30b, each designed to launch, receive, and/or reflect the acoustic wave. The response of piezoelectric biosensors is frequency dependent. This device provides flexibility that allows the designer to utilize the frequency that is optimal to measure the biological target in each channel separately. Different bioreceptor layers are deposited in the microfluidic channels. A bioreceptor layer 22a in FIG. 2A represents a specific bioreceptor layer that has been deposited on the sensing side 30a opposite the active electrical side 30b on which the acoustic generation and detection elements are found. FIG. 3 shows a cross sectional view of a packaged biosensor chip 30 capped with a compliant cover 40 that seals to the top of the walls 44 between and around the channels 26a, 26b, and also provides fluid connections 42 to off-chip instrumentation. The biosensor chip 10 is mounted in a surface mount package 46, and sealing material 48 is used to seal the cavity under the chip 10. The chip 10 can be mounted using gold bump bonding, for example.

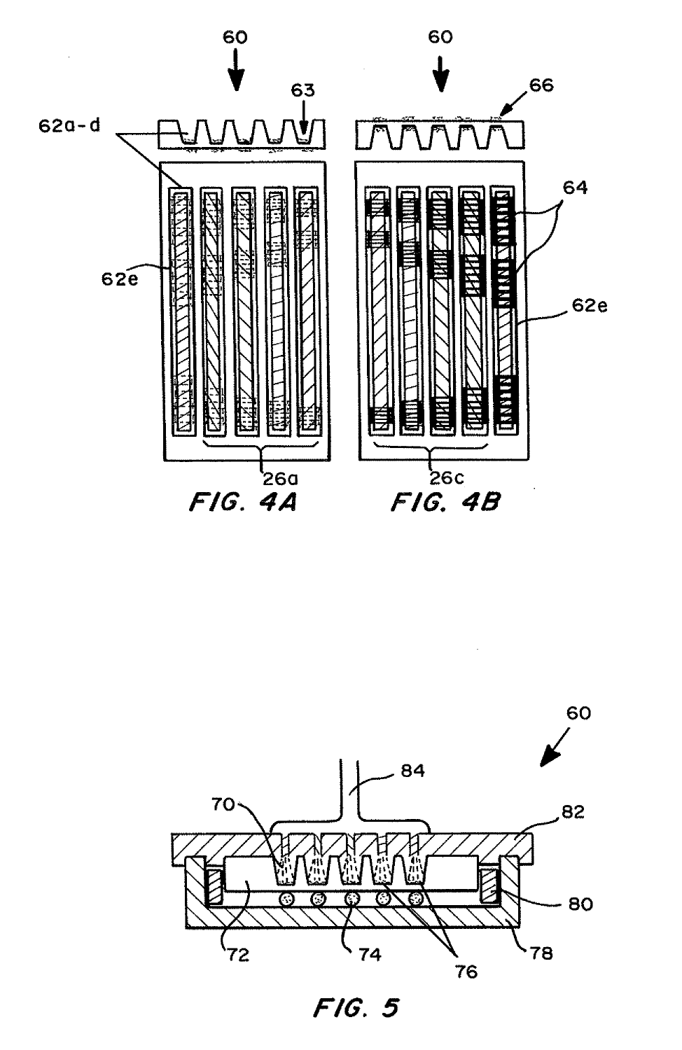

In another embodiment, a biosensor can have as many multiple channels etched into its substrate as needed. There is no limit to the number of biosensors, although there may be an optimal size before size becomes too large with the biosensors and the microfluidic components. An example of such a multiplexed biosensor is described in FIGS. 4A, 4B and 5 with reference to a sensor for the four serotypes of Dengue viral infection. The biosensor device 60 for detection of the dengue virus has four sensing channels 62a, 62b, 62; 62d (the fifth 62e is a reference channel), each with a single channel functionalized to detect one of four Dengue serotypes. Such a multiplexed design can either be engineered together on one integrated chip or consist of thin channels etched/cut into a single chip. FIGS. 4A and 4B show both a cross sectional front (electronic) view of the sensor chip (FIG. 4A) and back (biological sampling) view (FIG. 4B) of the sensing side of the sensor chip. FIG. 5 illustrates the cross-sectional view of a five channel chip. Sample is applied to the bioreceptor layer 63 in the channels 62a-d, then the metal electrodes 66 applied. Frequency detectors are located in the reference channels 62e.

During manufacture, a surface coating is added onto the chip through the common plenum as diagramed in the cross-section of the device 60 shown in FIG. 5. After channel functionalization by application of bioreceptors 70 into channels 76 is complete, a buffer solution may be introduced into the microfluidic channels 76 for chip encapsulation in a package 78 prior to use. The compliant cover 82 may then be replaced with a sealed 80 permanent cap and plastic cartridge packaging for device shipment, storage, and use. Alternatively, the compliant cover 82 can be made to have different fluid connections at the two ends of the channels as shown in FIGS. 3 and 5. In this configuration, the cap has separate fluid connections to each microfluidic channel at one end of the chip, and a common inlet plenum 84 at the other end. This cap configuration allows a microfluidic channel whereby the clinical samples are fed to the active and reference channels in parallel.

In some embodiments, the cap may be used both for functionalization during manufacturing and then flipped to load a clinical sample during sample testing. In this embodiment, solutions can be introduced through the individual channel feeds with waste exiting the device through a common plenum during manufacturing, and then the sample is introduced through the common plenum and waste will exit through individual channel fluid connections to the waste container added to the entire testing cartridge. This approach can also be implemented using micromachining and wafer-scale packaging techniques. Gold-gold bonding can be used to provide both the wafer bonding method and a compliant material for fluid channel sealing. Principal criteria used to determine the success of packaging development is the ability to (a) assemble device without breakage, (b) obtain proper electrical performance, and (c) achieve leak-free fluid flow through the device.

As can be seen in FIGS. 2-5, the biosensor chip is flip-chip mounted in a surface mount package, such as a standard ceramic surface mount package, using gold bump bonding for electrical die attachment. A sealing material such as room temperature vulcanizing silicon rubber (RTV) is placed around the edge of the biosensor chip to seal the biosensor chip to the surface mount package for added mechanical stability, and to ensure no liquid leakage to the area beneath the biosensor chip (with the electrical connections) occurs during chip manufacture or sample introduction.

The multiple channels etched into its substrate surface are subject to the operational limits of the electronic components and the dimensions of the microfluidic components that are governed largely by sample volumes. Referring to FIGS. 2, 3, 4, and 5, each channel 76 has multiple metal electrode structures 74 on the active electrical side, each designed to launch, receive, and/or reflect the acoustic wave. The response of piezoelectric biosensors is frequency dependent, providing flexibility to utilize the frequency that is optimal for detecting the biological target in each individual channel. Different bioreceptor layers are deposited in the microfluidic channels. A bioreceptor layer 22 in FIG. 3 represents a specific bioreceptor layer that has been deposited on the sensing side 30a opposite the active electrical side 30b on which the acoustic generation and detection elements are located. FIG. 3 shows a cross sectional view of a packaged biosensor chip 10 capped with a compliant cover 40 that seals to the top of the walls between and around the channels 26a, 26b, and also provides fluid connections to off-chip instrumentation. The biosensor chip 10 is mounted in a surface mount package 46, and sealing material 48 is used to seal the cavity under the chip. The chip can be mounted using gold bump bonding, for example.

C. Immobilization of Reactive Intermediates and Binder Molecules on Surfaces

For piezoelectric biosensors, surface activation is required before immobilization of the binding ligand or bioreceptor. This is often accomplished using self assembled monolayer (SAM) formation. Silanization via heterobifunetional silanes, in one example, 3-aminopropyltriethoxysilane (APTES)), has been used on bare piezoelectric substrates to provide modified surfaces with free amino groups suitable for covalent attachment of certain bioreceptors. Proteins, such as Protein A, Protein G and avidins such as strepavidin and neutravidin, also provide a convenient method for oriented immobilization of antibodies. Other non-covalent binding techniques can also be used as the first layer on bare lithium niobate surfaces.

Methods for conjugating antibodies onto surfaces are well known. Linkers of different lengths can be used to bind the antibody to the surface and can maximize binding strength, the minimal length being about 1 mm. A more flexible link will function well even if relatively short, while a stiffer link may need to be longer to allow effective contact between antibody and the link to the surface.

The length of a link refers to the number of atoms in a continuous covalent chain between the attachment points on the substrate and the binder molecule. Due to flexibility of the linker, all of the links may not have same distance from the surface. Thus linkers with different chain lengths can make the resulting binder more effective. Branched linkers bearing multiple functional groups also allow attachment of more than one binder molecules. The preferred lengths for linkers are 10, 15, 25, 30, 50, and 100 atoms or about 1 to 30 nm.

Hydrophilic or water-solubility linkers can increase the mobility of the attached antibody in aqueous media. Examples of water-soluble, biocompatible polymers which can serve as linkers include, but are not limited to polymers such polyethylene oxide (PEO), polyvinyl alcohol, polyhydroxyethyl methacrylate, polyacrylamide, and natural polymers such as hyaluronic acid, chondroitin sulfate, carboxymethylcellulose, and starch. Preferred forms of branched tethers are star PEO and comb PEO. Star PEO is formed of many PEO “arms” emanating from a common core.

The parking area (PA)or the projected area of the x-y dimensions of the linker onto the surface is another critical parameter, since it determines the maximum number of molecules in a given monolayer, ranging from about 1-2 nm squared for a linear silane to about 25 nm squared for streptavidin and about 200 nm squared for an IgG antibody. In contrast, a bound viral particle of 50 nm diameter occupies 2500 nm squared and a pathogen of 1000 nm, about 1 million nm squared. The PA for a binder like IgG dictates the optimum IgG coverage on the surface. High IgG loadings tend to interfere with conformational changes essential for optimal antibody interactions with antigen or receptors on target pathogens, thereby reducing binding affinity, capture rates and binding stability of target analytes.

Antibodies or other ligands, or linkers for antibodies or ligands, may be directly or indirectly covalently bound to chip surfaces by any functional group (e.g., amine, carbonyl, carboxyl, aldehyde, alcohol). For example, one or more amine, alcohol or thiol groups on the antibody may be reacted directly with isothiocyanate, acyl azide, N-hydroxysuccinimide ester, aldehyde, epoxide, anhydride, lactone, or other functional groups incorporated onto the surface of the device. Schiff bases formed between the amine groups on the antibody and aldehyde groups of the device can be reduced with agents such as sodium cyanoborohydride to form hydrolytically stable amine links (Ferreira et al., J. Molecular Catalysis B: Enzymatic 2003, 21, 189-199). Alternatively, the free amino groups of the antibody or binder proteins, like streptavidin or neutravidin, can be linked to a niobate or silica surface, e.g. by means of epoxide functional groups on 3-glycidoxypropyl trimethoxysilane.

The preferred reagents for depositing a reactive first monolayer on lithium niobate as the biosensor or chip are heterobifunctional silane reagents, such as 3-glycidoxypropyl trimethoxysilane (GOPS), 3-mercaptopropyl trimethoxysilane (MOPS), 3-aminopropyl triethoxysilane (APTES). The trimethoxysilanes are generally less reactive than triethoxysilanes. For GOPS, the silane portion is first conjugated to reactive hydroxyl groups on the niobate surface preferably as a monolayer. The glycidoxy (aka epoxy or oxirane) groups are then reacted at about pH 9-9.5 with up to 12 of the 24 available nucleophilic amino groups on one half of the neutravidin. In contrast, reaction of the epoxide functional groups with hydroxyl groups requires higher pH conditions, usually in the pH range of 11-12. Amine nucleophiles react at more moderate alkaline pH values, typically needing buffer environments of at least pH 9. Thiol groups, e.g. as on reduced IgG subunits, rapidly react at pH 7-8 (GT Hermanson in Bioconjugate Techniques, 1996, page 142).

In a preferred mode, the antibody or binder is coupled to the substrate surface by the use of a heterobifunctional silane linker reagent, or by other reactions that activate functional groups on either the surface of the substrate and/or the antibody. In general, immobilization of the antibody or binder using short linkers is generally non-oriented, often resulting in some loss of binding capacity and/or affinity. For example, carbodiimides as zero length linkers mediate the formation of amide linkages between a carboxylate and an amine or phosphoramidate linkages between phosphate and an amine. Examples of carbodiimides are 1-ethyl-3-(3-dimethylamino-propyl)carbodiimide hydrochloride (EDC), 1-cyclohexyl-3-(2-morpholino-ethyl)carbodiimide (CMC), dicyclohexyl carbodiimide (DCC), diisopropyl carbodiimide (DIC),

The preferred coupling mode of the antibody to the substrate surface involves a heterobifunctional linker or spacer. The linker may have both terminal amine and thiol reactive functional groups for reacting amines on substrates with sulfhydryl groups on subunits of antibodies, thereby immobilizing the antibody onto the surface in an oriented way. These linkers may contain a variable number of atoms. Examples of such links include, but are not limited to, N-Succinimidyl 3-(2-pyridyldithio)propionate (SPDP, 3- and 7-atom spacer), long-chain- SPDP (12-atom spacer), (Succinimidyloxycarbonyl-a-methyl-2-(2-pyridyldithio) toluene) (SMPT, 8-atom spacer), Succinimidyl-4-(N-maleimidomethypcyclohexane-1-carboxylate) (SMCC, 11-atom spacer) and Sulfosuccinimidyl-4-(N-maleimidomethyl)cyclohexane-1-carboxylate, (sulfo-SMCC, 11-atom spacer), m-Maleimidobenzoyl-N hydroxysuccinimide ester (MBS, 9-atom spacer), N-(g-maleimidobutyryloxy)succinimide ester (GMBS, 8-atom spacer), N-(g-maleimidobutyryloxy) sulfosuccinimide ester (sulfo-GMBS, 8-atom spacer), Succinimidyl 6-((iodoacetyl)amino)hexanoate (SIAX, 9-atom spacer), Succinimidyl 6-(6-(((4-iodoacetyl)amino)hexanoyl)amino)hexanoate (SIAXX, 16-atom spacer), and p-nitrophenyl iodoacetate (NPIA, 2-atom spacer). One ordinarily skilled in the art also will recognize that a number of other coupling agents or links, with different number of atoms, may be used.

Hydrophilic spacer atoms may be incorporated into the link to increase the distance between the reactive functional groups at the termini. For example, polyethylene glycol (PEG) can be incorporated into sulfa-GMBS. Hydrophilic molecules such as PEG have also been shown to decrease non-specific binding (NSB) and increase hydrophilicity of surfaces when covalently coupled.

In other embodiments, the free amine groups of the antibody are attached to a surface containing reactive amine groups via homobifunctional linkers. Using this chemistry, there is no control in antibody orientation. Linkers such as dithiobis(succinimidylpropionate) (DSP, 8-atom spacer), disuccinimidyl suberate (DSS, 8-atom spacer), glutaraldehyde (4-atom spacer), Bis[2-(succinimidyloxycarbonyloxy)ethyl]sulfone (BSOCOES, 9-atom spacer), all requiring high pH, can be used for this purpose. Examples of homobifunctional sulfhydryl-reactive linkers include, but are not limited to, 1,4-Di-[3′-2′-pyridyldithio)propion-amido]butane (DPDPB, 16-atom spacer) and Bismaleimidohexane (BMH, 14-atom spacer). For example, these homobifunctional linkers are first reacted with a thiolated surface in aqueous solution (for example PBS, pH 7.4), and then in a second step, the thiolated antibody or protein is joined by the link.

The tedious sequential multi-step conjugation method for functionalizing a sensor surface, described in Ben-Dov, et al. in Anal. Chem. 69(17):3056-3012 (1997) has limited practical or commercial value. Other binding approaches, such as direct binding of antibodies to thiol, amine, or carboxylic acid functional groups on self assembled (SAM) monolayer have been used to produce films which exhibit large fractional changes in mass with viral binding, resulting in enhanced sensitivity.

The most preferred mode, found to be superior to alternative binding modes, involves streptavidin or neutravidin that is immobilized or bound to the lithium niobate surface to form a biosensor film via a variety of methods for derivatizing the surface, e.g. silanization with a heterobifunctional silane. The reported affinity of biotin for the avidins is in the femtomolar range or about one million times higher than that of the typical antigen-antibody interaction. Hence both capture kinetics and binding stability are substantially higher for targets bearing biotinylated antibodies or binders, both factors being essential in achieving short incubation times and minimal washoff from dissociation that are critical in rapid POC tests. Binding multiplicity from multiple biotin moieties on the target interacting with the immobilized neutravidin in the contact area provides a further exponential increase in binding avidity for the avidin capture surface, approaching “infinite affinity”, a theoretical concept first proposed by Claude Meares et al (The Chemistry of Irreversible Capture, Adv Drug Delivery Rev. 60, 1383-1388 (2008)).

The strength of multiple binding interactions is critical in minimizing dissociation or washoff of relatively large micron sized target cells linked via a limited number of covalent bonds to the niobate surface. Such losses may occur from hydrodynamic stresses during flow through or buffer washes. Affinity enhancement in a multivalent binding mode also applies, albeit to a far lesser extent, to the interactions of antigen/epitopes with the corresponding immunoreactive antibody/binder pairs.

Selected SAM-based base layers will be utilized on both bare piezoelectric and on Au coated piezoelectric to provide amine, carboxylic acid, or other appropriate binding sites for the antibodies. Direct binding of anti-N. gonorrhoeae and anti-C. trachomatis antibodies to SAM layers will be evaluated for feasibility, as this direct binding may produce films that exhibit the largest fractional change in mass with bacterial binding, and hence have the greatest sensitivity. Silanization, protein-based, and alkanethiol based SAM films will be considered with appropriate functionalizations. Nanostructured oligo(ethylene-glycol) films will be given particular attention, due to their ability to reduce nonspecific binding. Modification of the nanostructure of such films to provide a beneficial distribution of binding sites for the antibodies will be performed. Once direct binding of anti-N. gonorrhoeae and anti-C. trachomatis antibodies to the device has been demonstrated, and baseline sensitivities determined for this approach, alternative methods to enhance response sensitivity will be evaluated, and the most promising of these will be developed and tested. The use of antibodies with multiple binding sites for the target analytes (as presented for C. trachomatis) will be evaluated, as will sandwich assays using both monoclonal and polyclonal antibodies. Finally, nanoparticle based films, incorporating functionalized nanoparticles, will be considered. The use of dendrimer-like macromolecules will also be evaluated.

D. Bioselective Binding Agents for Use in Biosensor Systems

A wide range of bioselective binding agents can be used on biosensor surfaces to bind and detect various biological molecules and pathogens in liquids. Antibodies, which naturally bind antigens (e.g. proteins, carbohydrates, small molecules), commonly with nanomolar affinities, have been used most widely for this purpose. Also useful are binding ligands with extremely high affinity, such as biotin for the avidin proteins, e.g. streptavidin and neutravidin with affinities in the femtomolar range, enabling coating of the biosensor surface with an avidin, thereby providing far more rapid capture of biotinylated targets, e.g. cells bearing biotinylated antibodies, than antibodies binding to target epitopes.

As used herein, unless otherwise specified, “antibodies” includes polyclonal, monoclonal, single chain, free subunits and antibody subunits or combinations thereof as substitutes for polyclonal antibodies. The antibodies can be xenogeneic, allogeneic, syngeneic, or modified forms thereof, such as humanized, chimeric antibodies or recombinantly created forms such as those selected by phage based technology.

As used herein, ligands such as complementary nucleic acid sequences and binding proteins can be utilized as well to detect antigens of interest. Alternative binding agents such as DNA and RNA aptamers, receptor proteins or ligands, both natural or synthetic, and/or nanomaterials may also be used.

The enhanced binding kinetics of biotin coated targets to immobilized avidins is important in rapid detection of low levels of pathogens in complex media like blood where diffusion is significantly impeded by a vast number of non-target cells, thereby prolonging incubation times that need to minimized in POC tests to get a result while the patient is still on-site.

Further advantages of a generic capture agent like immobilized neutravidin are: defined protein with multiple amino functional groups for conjugation to amine reactive chip coatings (e.g. heterobifunctional silanes), availability in large quantities as a uniform raw material at relatively low cost, cost reductions in manufacturing, quality control and inventory maintenance of a chip with a generic coating, and high thermal stability of neutravidin during storage at elevated temperatures that is highly important for POC testing in tropical climates. Pretreatment of the sample with biotinylated antibody requires storage and reconstitution in dry state, either on-board or off-board, prior to flow through and capture of the biotinylated target by the immobilized neutravidin film in a target specific channel.

The perceived disadvantage of dry storage of potentially labile capture antibodies can be obviated by addition of protein stabilizers, surfactants, lysing agents, blockers of NSB and/or other sample pre-treatment agents in a dry cocktail that is reconstituted with the specimen. The concurrent off-chip incubation inside the antibody storage well or tubing provides rapid and full coating of all target epitopes with biotinylated antibodies before subsequent rapid capture by flow-through of the biotin labeled targets in the designated neutravidin channel.

The excess biotinylated antibody captured on neutravidin in the absence of or at low levels of target entities increases the layer thickness that increases the baseline or background signals in SAW detection, but the effect can be readily compensated in the data reduction algorithm.

Biotinylated fluorescent beads of about 1.1 micron diameter provide an excellent model for studying capture and kinetics of biotin-antibody labeled cells on immobilized neutravidin layers by means of fluorescence microscopy. The fluorescein-loaded beads can be readily detected in the FITC channel down to a level of 1 to 2 beads using serial dilutions of stock solutions in PBS-BSA-Tween® 20.

The excellent results with the fluorescent bead model were confirmed with comparable studies using inactivated Elementary Bodies (EB) from Ch. trachomatis and biotin labeled antibodies detectable after nuclear staining with DAPI.

To test the sensor, antibodies to Chlamydia trachomatis were linked to biotin and subsequently allowed to react with immobilized neutravidin on the biosensor surface. The results demonstrates that Chlamydia EB binding is specific, since no evidence of binding is observed when no biotinylated antibody is present and when a non-specific antibody is bound to the chip surface.

Experiments with neutravidin coated biosensors show that lithium niobate is capable of specifically binding model beads of biotinylated fluorescent latex particles (1.1 μm diameter) and biotinylated Elementary Bodies (EB) of inactivated Chlamydia trachomatis with a high signal to noise ratio.

In one embodiment, the chips are prepared as described above and instead of binding with Chlamydia EBs, Dengue virus is bound to the lithium niobate wafer. Since viruses are smaller than EBs, the detection limits of the SAW will demonstrate that smaller biological specimens can be detected in the proposed system.

Antibodies specific to each serotype of Dengue Virus (DEN1, DEN2, DEN3 and DEN4) have been characterized in the literature. All four are mouse monoclonal antibodies against type specific determinant on Dengue viruses.

These antibodies are:

Anti-Den1:15F3-1 (ATCC No HB-47)

Anti-Den2:3H5-1 (ATCC No HB-46)

Anti-Den3:D6-8A1-12

Anti-Den4:1H10-6-7

The monoclonal antibodies are purified using standard methods such as a protein A column.

Anti-Den1:15F3-1 (ATCC No HB-47) and Anti-Den2:3H5-1 (ATCC No HB-46) are described in Henchal et al., Am J Trop Med Hyg, 31:830-6 (1982). Anti-Den3:D6-8A1-12 and Anti-Den4:1H10-6-7 are described in Tewari et al., Trop Med Int Health, 9:499-507 (2004). They are specific for virus serotypes (Ansarah-Sobrinho, et al., Virology. 381:67-74 (2008). Accordingly to the CDC, they also do not cross react with other flaviviruses.

These dengue virus serotype-specific antibodies or other suitable reagents are used to functionalize the biosensor chip.

Before binding the four monoclonal antibodies (Anti-Den1:15F3-1 (ATCC No HB-47), Anti-Den2:3H5-1 (ATCC No HB-46), Anti-Den3:D6-8A1-12, and Anti-Den4:1H10-6-7) or antibodies raised using methods known to one of ordinary skill in the art, to the biosensor chip, their specificity and sensitivity toward their cognate Dengue Reporter Virus (DRV) is determined by ELISA.

Briefly, DRVs are adsorbed on 96-well plates and then blocked with 2% BSA. The plates are incubated with serial dilutions of monoclonal antibodies and then alkaline phosphatase-conjugated anti-mouse IgG, and p-nitrophenyl phosphatase. The OD value is read in a plate reader at 405 nm. ELISA is used to confirm the specificity of the monoclonal antibodies to their cognate virus. The limit of detection of the virus by ELISA serves as comparison for the performance of biofunctionalized chip.

Multi-channel biosensor chips will be tested as part of clinical studies in an approved biohazard facility against known patient samples (positive and negative) for sensitivity and specificity. The known samples will include purified DEN virus and a collection of defined clinical blood samples and selected potential interferring agents to demonstrate selective detection. Mock clinical samples can be purchased from a vendor. For example, for testing for Dengue virus (DV), samples consist of DV at various concentrations in PBS, or other buffer as fitted, with pure carrier as a negative control.

Based on the results of tests with buffer-based samples, a collection of mock clinical samples will be produced and tested. Mock clinical samples will consist of DV in human blood, plasma or serum and, if needed, diluted in PBS buffer. Samples that consist of PBS with the target virus at varying concentration levels will be tested, with pure PBS as a control.

Additional testing with other potential contaminants will be conducted to verify selectivity. For example, non-DEN flaviviruses will be evaluated for NSB. The results of these tests will demonstrate specificity and provide estimates of sensitivity.

E. Methods to Reduce Non-Specific Binding

Non-specific binding (NSB) of non-target species to immobilized binders on surfaces is a common interference in immunoreactions between antigens and antibodies. Both specific binding and NSB are caused mainly by weak complementary hydrophobic and/or ionic interactions that increase exponentially with the number of interactions. NSB differs from specific binding in having about 3-5 log lower affinities than antibodies, largely due to fewer favorable interactions. Hence NSB can often be reduced or inhibited by a combination of proteins, surfactants, changes in pH, buffers that minimize or prevent such interactions or by simple dilution. Blocking proteins include plasma proteins, albumins, fat free milk, gelatin and other materials used for this purpose. Surfactants include Tween® 20, Triton® X-100, PEG, Pluronic® F68 and F127, etc. Commercial proprietary blocking formulations are available for specific applications and are often more effective than bovine serum albumin (BSA) or casein in fat free milk.

Reduction of nonspecific binding of non-target components likely to be found in samples is important in order for the sensor to be able to perform with the highest possible sensitivity. NSB must be minimized since it can affect baseline responses in SAW detection, adversely impacting the limit of detection (LOD) and spuriously elevate levels of target entities. Numerous studies have addressed the use of SAMs to enhance resistance to nonspecific adsorption of specific proteins and cells, or to promote the binding of specific proteins (Otsuka, et al., Current Opinion in Colloid and Interface Science, 6:3-10 (2001); Chapman, et al., J. Am. Chem. Soc., 122:8303-8304 (2000); Otsuni, Langmuir, 17:6336-6343 (2001); Chapman, et al., Langmuir, 16:6927-6936 (2000)). Specifically poly (ethylene glycol) (PEG) has been shown to provide specific binding of target proteins while minimizing nonspecific adsorption of other proteins (Otsuka, et al., Current Opinion in Colloid and Interface Science, 6:3-10 (2001)). Such films can be used to reduce nonspecific binding. It is also possible to include reagents such as a hydrophilic polymer like polyethylene glycol (“PEG”) or a surfactant such as Tween® 20 in the wash buffer. These can improve wash efficiency when the wash buffer is applied or allowed to flow over the sensor to remove the non-specifically bound agents.

Reduction of nonspecific binding of other components likely to be found in samples is important in order for the sensor to be able to perform with the highest possible sensitivity. Numerous studies have addressed the use of SAMs to enhance resistance to nonspecific adsorption of specific proteins and cells, or to promote the binding of specific proteins. Nanostructure formation from block copolymers, most specifically polyethylene glycol) (PEG) has been shown to provide specific binding of target proteins while minimizing nonspecific adsorption of other proteins. The unique macromolecular structure of the film formed from block copolymer micelles with cross-linking cores, and the PEG functionalized surface in brush form should be used. PEG chains tethered to the surface of the nanometer-scaled micelles described should be able to provide steric exclusion of other large molecules and particles from binding, thus preventing proteins and cells from adhering to the surfaces. Such nanostructured films hold great promise for development of sensors that will be in contact with blood and urine samples.

There are several film characteristics that are significant to successful biosensor development, in addition to those related to biological specificity and sensitivity. Specifically, the film adhesion, robustness, reproducibility of film deposition, compatibility with processing steps that will be used following film deposition, and device aging and insertion loss increases caused by the films should be tested. All films evaluated will also be tested for changes in device performance with film deposition and liquid loading. These tests will be electrical tests using an Agilent E5070B network analyzer, and data obtained will be compared with baseline electrical performance to determine the effects of the film. For films that demonstrate promising from a biological performance perspective, additional characterization will be performed.

F. Sample Delivery to the Sensor System on the Testing Cartridge

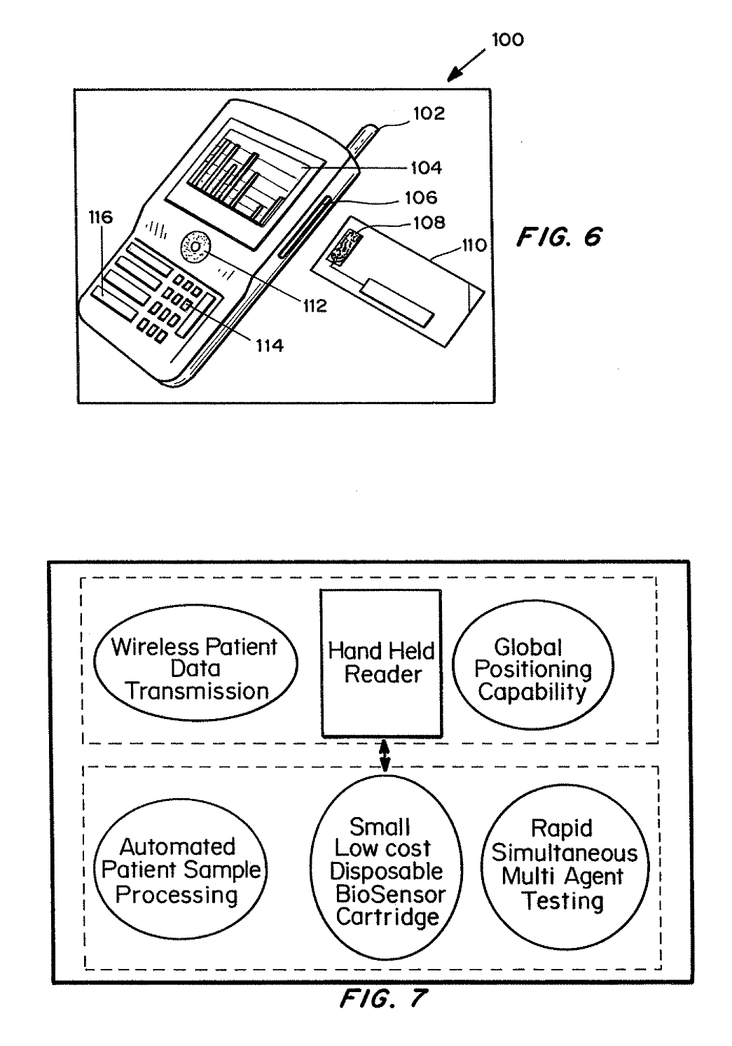

During system manufacture, the compliant cover of the chip is connected to multiple fluid sources in order to functionalize each channel with the specific biosensor receptors needed to detect the target serotype of interest in that channel as shown in FIG. 5. The handheld reading system is shown in FIG. 6 and a general schema of the testing system is shown in FIG. 7. The testing cartridge is developed from the start taking into account device geometry, packaging, and handling issues in order to allow automation of this process, using industry standard processes, for high volume manufacturing.

The sample for testing may be blood, plasma, serum, lymph, mucous secretion, tissue, interstitial fluid, fecal materials, mucus, tears, tissue exudates, urine, saliva, or other body fluids. The test system has multiple but separate intake wells where different samples or aliquots of the same sample can be applied. Although in this rendition the intake wells lead directly to the capillary action wells, it is possible to insert a filter just before the capillary channels to separate large particulate matter such as cells or debris from the clinical sample. Such filtering devices are available in the marketplace from specialized microfluidics companies.

Clinical samples are obtained using standard techniques. Blood may need to be lysed or otherwise separated in a collection container to release viral particles or viral proteins or separated on the microfluidic channel utilizing specific membrane or other separating agents/films/channels. Initial calculations indicate that a finger stick should provide sufficient sample for analysis. The estimate for the amount of sample required for diagnosis is 1.0 ml or less. The sample can be manually transferred from either a common blood collection tube or specialized capillary that can perform a finger stick to the chip. Optionally, the blood may need to be treated with nucleases and anti-coagulant reagents (EDTA, heparin) to prevent changes in the fluidic properties (i.e., viscosity) which may impair the ability of the fluid to move over the sensor. Correct treatment of the specimen can also minimize non-specific binding. The sample may be treated in the collection device or in the cartridge itself.

G. Hand-Held Detector and Integrated POC System

An integrated microfluidic system is shown in FIG. 6. This includes a device 100 into which the biosensor cartridge 110 is inserted. The fluidic system is integrated with the biosensor cartridge 110 as the detecting sensor 108 (SAW), forming a Lab-on-a-Chip structure which is inserted into the hand-held device slot 106. The system is designed for two step operation: sample processing and flushing with buffer. Channel geometry, hydrophilic and hydrophobic properties of the micro-fluid system are optimized for maximal target attachment in the immobilization zone.

Methods for ensuring that the amount of fluid sample applied to a device is sufficient prior to conducting a desired test, as well as methods for controlling the flow of fluid through a device, are disclosed in U.S. Pat. No. 5,234,813 to McGeehan, et al. and U.S. Pat. No. 6,759,009 to Law.

A point of care detection system is designed to perform the following functions: 1) guide the user through the procedural steps, 2) allow automated rapid analysis of a specimen from sample introduction to read sensor output, 3) communicate result to the operator, 4) convey the information to medical experts such as physicians, public health agencies in a timely fashion using modern wireless/cellular mobile systems incorporating features such as Global Positioning Systems and/or BlueTooth wireless systems 102. These capabilities are illustrated in FIG. 7.

In addition to basic architecture, other functions such as buffer release and waste systems can also be incorporated into the fluidic design and into the final test kit. In a preferred embodiment, a 32 bit PIC microcontroller design/evaluation kit is used. The microcontroller has basic input-output capabilities including touch-screen interface, navigation, numeric and analytic keypads 112, 114, 116, USB communication, SD card data storage.

Power management includes a battery, charger (solar and non solar) and external power supply (DC or AC). The input signal from the SAW device will be preconditioned (amplified, compensated for temperature and filtered if necessary). The microcontroller provides a powerful tool for crucial tasks such as self testing and calibration, result storage, procedure guide and control.

The present invention will be further understood by reference to the following non-limiting examples.

### EXAMPLE 1

### Functionalization of Microchannels

Functionalization of the channel surfaces is necessary for piezoelectric affinity biosensors to selectively bind target analytes. The preferred mode is a 2-layer approach using 3-glycidoxypropyl trimethoxysilane (GOPS) in a first step to activate the chip surface, followed by functionalization with neutravidin to provide the capture surface for biotin-antibody labeled target entitities.

The preferred beads as models of target cells for detecting functionalization and monitoring of biosensor surfaces bearing neutravidin coating are fluor loaded latex beads also bearing biotin (Invitrogen, F-8768, exc/em 505/515 nm; 1.1 micron size containing high loadings of fluorescein). Such beads were used in optimizing the coating chemistries and conditions of the niobate chips.

The silanes, 3-glycidoxypropyl trimethoxy silane (GOPS) and 3-mercaptopropyl trimethoxy silane (MOPS; both from Gelest. PA), were used as the base layers initially on glass microscope slides serving as model surfaces for niobate by dipping into a 0.1% solution in 2-propanol-water (9:1), removal of excess fluid with nitrogen gas and baking for 15 min at about 100° C. Covalent conjugation of GOPS surfaces was done with neutravidin (Invitrogen; 0.05 mg/mL, pH 9.5 in 10 mM carbonate for about 1 hr) followed by removal of unbound neutravidin with Tris buffer, pH 7.0 and drying. MOPS conjugation was used to form maleimido-neutravidin (Invitrogen; in PBS at pH 7.4 for ½ hr) followed by rinsing with PBS.

Prior to testing with biological target cells, exploratory tests were performed with 0.1 M phosphate buffered saline (PBS) containing 0.1% Tween® 20 and biotinylated fluorescent calibration beads of defined size to establish binding characteristics of neutravidin functionalized biosensors. Such biotin bearing latex beads are functionally equivalent to target cells bearing biotinylated antibodies and thus serve as model cells for optimizing the binder chemistries on niobate chips and testing capture kinetics from various chamber configurations prior to proceeding to detection by SAW. The same methods described above were used to provide functional silane layers for covalently attaching neutravidin and maleimido-neutravidin to the surface of the niobate biosensors (5×10 mm) as demonstrated by specific binding of biotinylated fluor latex beads diluted to about 10,000 beads per microliter in PBS. These were easily imaged and enumerated on an inverted fluorescent microscope (Zeiss Axiovert). As few as 1-2- beads are readily detectable at 100× magnification. These beads are also highly resistant to washoff or photobleaching at the excitation wave length of about 495 nm.

### EXAMPLE 2

### Functional and Biological Testing of Chip Surfaces

Biological target cells of elementary bodies (EB) of inactivated C. trachomatis were captured on antibody coated chip surfaces and stained using fluorescent staining with two specific stains: the nuclear stain DAPI and a fluorecein labeled anti-mouse IgG for a Mab labeled epitope of EB. The purpose was to demonstrate specific capture of EB, labeled with both DAPI and fluorescein, on chip surfaces, to establish the limit of detection by titering and to assess specificity as seen in low fluorescence from NSB. These studies were done prior to performance studies in the SAW detection mode. Binding to the chip was demonstrated when low or high concentrations of EB were added.

The selected antibodies will be bound to prototype biosensor chips using the processes appropriate for the nano-film and antibodies being evaluated. These prototype coated chips will be used for further characterization of the bioselective films, and for testing with known bacterial samples and mock clinical samples (known bacteria in human urine).

### EXAMPLE 3

### Selective Detection of Inactivated C. Trachomatis Using Spiked Samples

Practical applications in POC clinical diagnosis frequently require rapid multiplexed test capability providing results for more than one condition from a single sample. The multiplexed biosensor chips were tested against inactivated purified strains of N. gonorrhoeae and C. trachomatis, along with a collection of mock clinical samples (known bacteria in human urine) and controls to demonstrate selective detection.

Multiplexed sensor array chips were tested against known bacterial samples (positive and negative) for sensitivity and specificity. Purified strains of inactivated N. gonorrhoeae and C. trachomatis, spiked in PBS, individually and in combination at several concentrations, were tested, with PBS as a control. The mass sensitivity of the biosensor devices of less than a picogram allows detection of low bacterial concentrations down to a single bacterium.

Based on the results of tests with PBS-based bacterial samples, a collection of mock clinical samples were prepared, using human urine and inactivated N. gonorrhoeae and C. trachomatis, spiked with appropriate concentrations of cultured microorganisms. Additional testing with inactivated N. gonorrhoeae and C. trachomatis and at least two other bacteria commonly involved in urinary tract infections or in vaginal infections such as Escherichia coli, Proteus mirabilis, Gardnerella vaginialis, Group B Streptococcus, Staphylococcus aureus and Enterococcus feacalis were also tested to verify selectivity.

Unless defined otherwise, all technical and scientific terms used herein have the same meanings as commonly understood by one of skill in the art. Less common or unique expressions used in this Application are appropriately defined. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. An integrated biosensor system comprising
a piezoelectric substrate with one or more microfluidic channels thereon,
the channels having one or more infectious agent analyte specific ligands or receptors bound thereto,
means for delivering flow of a fluid sample through the channels,
wherein a reaction between the ligand or receptor and the infectious agent analyte causes a detectable change in the surface acoustic wave properties in the channels of the piezoelectric substrate.

2. The biosensor system of claim 1 further comprising means for delivery of reagents to the channels.

3. The biosensor system of claim 1 wherein the piezoelectric substrate having channels thereon comprises a disposable cartridge comprising fluidic handling means.

4. The biosensor system of claim 1 further comprising an analytical reader.

5. The biosensor system of claim 4 wherein the analytical reader is a portable and self contained device with wireless or cell phone capability enabling transmission of test results to a central data processing center.

6. The biosensor system of claim 1 capable of processing fluid samples and providing test results within a few hours.

7. The biosensor system of claim 1 wherein the ligand or receptor is an antibody for detection of infectious agents.

8. The biosensor system of claim 1 comprising immobilized avidin, neutravidin, or streptavidin and biotinylated antibody or fragment thereof.

9. The biosensor system of claim 1 wherein the infectious agent is a pathogen of viral, bacterial, or parasitic origin, or component or product.

10. The biosensor system of claim 9 wherein the infectious agent has more than one serotype or serovar.

11. The biosensor system of claim 9 wherein the agent exists as one or more strains.

12. The biosensor system of claim 1 wherein the channels have a size range from 100×100 mm to 5×15 mm.

13. The biosensor system if claim 1 wherein the substrate can accommodate two to six 6 separate channels.

14. The biosensor system of claim 1 wherein the channel widths are from 0.1 to 5 mm, have a length from 1 to 100 mm, and can accommodate sample volumes of 1 to 1000 μL.

15. The biosensor of claim 1 comprising a piezoelectric substrate selected from the group consisting of tantalate, silica and lithium niobate (LiNbO₃).

16. The biosensor system of claim 14 wherein the piezoelectric substrate comprises a 0.05-0.5 mm thick LiNbO₃ wafer.

17. A method for detecting the presence of an infectious analyte comprising
obtaining a biological sample fluid,
delivering a sample fluid into one or more channels of a biosensor piezoelectric substrate as defined by claim 1, and
detecting binding reactions with one or more infectious analytes by means of detectable changes in the surface acoustic wave properties in the channels.

18. The method of claim 17 wherein the biological sample fluid is blood, a blood fraction, lymph, sputum, urine, fecal material, saliva, mucous, tears or a tissue exudate.

19. The method of claim 17 wherein the analyte is selected from the group comprising bacteria, virus, parasites, products thereof, and components thereof.

20. The method of claim 19 wherein the analyte is strain specific.

21. A method of making the biosensors of claim 1 comprising immobilizing analyte-specific ligands or receptor reactive with an infectious agent, component or product thereof in the channels of the biosensor.

22. A handheld device providing a point of care detection system comprising
A biosensor comprising a piezoelectric substrate with one or more microfluidic channels thereon,
the channels having one or more infectious agent analyte specific ligands or receptors bound thereto,
means for delivering flow of a fluid sample through the channels,
wherein a reaction between the ligand or receptor and the infectious agent analyte causes a detectable changes in the surface acoustic wave properties in the channels of the piezoelectric substrate,
means for automated rapid analysis of a specimen from a sample introduced into one or more channels of the biosensor,
means for reading sensor analysis output,
means for communicating or displaying the results of the analysis,
means for conveying the information wirelessly or through a usb type port.

23. The device of claim 22 further comprising input-output capabilities selected from the group consisting of touch-screen interface, navigation, numeric and analytic keypads, USB communication, and SD card data storage and a microcontroller for self testing and calibration, result storage, procedure guide and control, and global positioning data.

24. The device of claim 22 further comprising power management.

25. Wirelessly transmitting the test results of claim 17 using a biosensor system comprising
a piezoelectric substrate with one or more microfluidic channels thereon,
the channels having one or more infectious agent analyte specific immobilized ligands or receptors bound thereto,
means for delivering flow of a fluid sample through the channels,
wherein a reaction between the ligand or receptor and the infectious agent analyte causes a detectable changes in the surface acoustic wave properties in the channels of the piezoelectric substrate, and
wherein the analytical reader is a portable and self contained device with wireless or cell phone capability enabling transmission of test results to a central data processing center.
